(19) United States
(12) Patent Application Publication
(10) Pub. No.: US 2026/0158495 A1
(43) Pub. Date: Jun. 11, 2026

(54) SYSTEM FOR THE AUTOMATED PREPARATION OF A BIOLOGICAL SAMPLE

(71) Applicant: BIOMERIEUX, Marcy L'Etoile (FR)

(72) Inventors: Jerome BOURDIN, Grenoble (FR); Laure FERNET, Grenoble (FR); Michel GUY, Grenoble (FR); Bruce HARRISON, Grenoble (FR)

(73) Assignee: BIOMÉRIEUX, Marcy L'Etoile (FR)

(21) Appl. No.: 18/707,981

(22) PCT Filed: Nov. 14, 2022
(86) PCT No.: PCT/FR2022/000107

§ 371 Date: May 7, 2024

(30) Foreign Application Priority Data
Nov. 15, 2021 (FR) FR2112060

(51) Int. Cl. B01L 7/00; B01L 9/00; C12Q 1/68

(52) U.S. Cl. CPC B01L 7/52; B01L 9/523; C12Q 1/68; B01L 2200/0689; B01L 2300/041; B01L 2300/0829; B01L 2300/1827; B01L 2300/1883; B01L 2300/1894

(57) ABSTRACT

A system for the automated preparation of a biological sample or of a biological mixture, including at least: a plate support; a plate including a body, from which a plurality of wells extends, the plate being at least partially housed in the plate support; at least one cooling module; wherein the plate is positioned on the cooling module so that each well of the plate is partially inserted into the cooling module, the body of the plate being at a predetermined distance from the cooling module so as to delimit a space between the body of the plate and the cooling module.

### TECHNICAL FIELD OF THE INVENTION

The invention relates to the technical field of in vitro molecular diagnosis. More specifically, the invention relates to the automated preparation of biological samples before “Polymerase Chain Reaction” (PCR) treatment.

### TECHNOLOGICAL BACKGROUND OF THE INVENTION

Various methods exist for identifying the presence and/or the nature of one or more microorganisms.

One of the methods that is used is a molecular biology method for in vitro gene amplification. This method is also called “Polymerase Chain Reaction” (PCR) based on the repetition of temperature transition cycles and generally involves a series of steps (denaturation, hybridization, elongation) implemented in a thermocycler instrument.

This molecular biology method in some cases can require upstream preparation of a biological sample in order to insulate the nucleic acids required for amplification. To this end, the preparation of the sample comprises at least one step of extracting nucleic acids contained in the biological sample. Once extracted, the nucleic acids are recovered in the form of eluate and are distributed in a plate comprising a plurality of wells for amplification, with the distribution being carried out by preferably automated pipetting.

One or more reagents is/are dispensed in each well of said plate for implementing the polymerase chain reaction method. In order to prevent the reaction from starting before incorporating the eluate, it is recommended that the reagents are maintained at a temperature ranging between 4° C. and 15° C.

One of the problems encountered while thus maintaining the temperature is condensation on the plate, which can thus cause contamination from one well to the next.

In the present invention, the term “biological sample” is understood to mean a suspension of biological agents or a mixture of suspensions of biological agents. The biological agents are, for example, microorganisms (bacteria, yeasts, mold, etc.). In the present invention, the term “biological mixture” is understood to mean a mixture of reagent and a biological sample or an eluate.

In the present invention, the term “eluate” is understood to mean a biological sample that has been treated or pre-treated in order to re-suspend purified and/or concentrated biological agents.

### SUBJECT MATTER OF THE INVENTION

The aim of the invention is to overcome all or some of the aforementioned disadvantages and notably to avoid the formation of condensation on the surface of the preparation plates.

To this end, the subject matter of the invention is a system for the automated preparation of a biological sample or of a biological mixture comprising at least one reagent and a biological sample or an eluate of the biological sample, comprising at least:
a plate support;
   a plate comprising a body, from which a plurality of wells extends that are configured to contain a reagent and/or a biological sample or a biological sample eluate containing at least nucleic acids, each well comprising an opening emerging onto the body of the plate and a bottom, each well longitudinally extending in a direction substantially perpendicular to a direction Z-Z′ along which a surface of the body of the plate extends, said plate being at least partially housed in the plate support;
at least one cooling module;
   characterized in that the plate is positioned on the cooling module so that each well of the plate is partially inserted into said cooling module, the body of the plate being at a predetermined distance d from the cooling module so as to delimit a space between said body of the plate and the cooling module.

This configuration allows the cooling of surfaces to be limited, and notably the surfaces of the body of the plate where condensation can form and become problematic. Furthermore, as the material used for manufacturing the plate has low thermal conduction, ranging between 0.1 Wm⁻¹K⁻¹ and 0.4 Wm⁻¹K⁻¹, the entire plate does not need to be cooled but only the zones where a heat exchange with the reagents is desired, i.e., a lower portion of each well.

According to one feature of the invention, the system comprises a peripheral seal, preferably made of elastomer, arranged in the space between the cooling module and the body of the plate, which avoids cold transmission by convection from the cooling module to the plate and circumscribes the zones to be cooled.

According to one feature of the invention, the space formed between the cooling module and the body of the plate is provided with a thermal insulator. Preferably, the thermal insulator is a fluid such as air, or a seal or a layer of insulating foam. Adding a thermal insulator allows the body of the plate to be thermally insulated relative to the cooling module.

According to one feature of the invention, the system comprises a vacuum chamber arranged between the body of the plate and the cooling module, which improves the thermal insulation of the body of the plate.

According to one feature of the invention, the system comprises at least one heating component positioned in the vicinity of the body of the plate and insulated from the cooling module.

According to one feature of the invention, the heating component is a resistive electrical film. Alternatively, the heating component is a plate made of ceramic or of a thermally conductive alloy engaging with the plate, and notably with the bottom of the wells, in a form-fitting manner.

Thus, the system has two distinct zones with a different temperature, with the first zone being located between the body of the plate and a thermal insulator or a peripheral seal and the second zone being located in the vicinity of the cooling module and the temperature in the first zone being higher than the temperature within the second zone.

The advantage of such a configuration with two distinct zones is to be able both to maintain the temperature of the reagents in order to limit their biological activity or their degradation, while keeping the body of the plate at a temperature above the dew point.

According to one feature of the invention, the cooling module comprises a first part configured to engage with a portion of the plate. Preferably, the first part of the cooling module engages with the lower portion of each well.

According to one feature of the invention, the first part of the cooling module has a plurality of cavities distributed over the surface of the first part, each cavity being shaped so as to partially accommodate a well of the plate.

According to one feature of the invention, the support comprises a housing configured to receive the plate.

According to one feature of the invention, the housing comprises a circumferential edge, on which part of the cooling module rests. Preferably, the first part of the cooling module is positioned on the circumferential edge of the housing of the support.

Advantageously, at least one wedging pin is arranged on the circumferential edge. This pin allows precise adjustment of the positioning along the Y-Y′ axis of the plate in order to reduce the dead volume, i.e., the volume of liquid remaining in the well that cannot be taken by the dispensing machine.

Preferably, the plate assumes a parallelepiped shape and the housing of the support assumes a complementary shape.

Preferably, the circumferential edge of the housing of the support has four corners, with a wedging pin being positioned in each of the corners.

According to one feature of the invention, the housing of the support further comprises at least one lug in the form of a protuberance extending in a direction perpendicular to the surface of the body of the plate. Advantageously, the at least one lug is configured to engage with a cover of the system in order to translationally block the plate in the housing of the support. Advantageously, the at least one lug provides guidance for introducing the plate into the cooling module. Furthermore, when removing the cover, the lugs guide the removal movement of the cover, which avoids touching the plate, which could be picked up by the cover causing shocks, the solutions to splash into the wells or could even cause the plate to fall and spill its contents.

According to one feature of the invention, the at least one lug is in the form of an extension of a corner of the housing of the support, which assists the lateral retention of the plate within the housing of the support.

According to one feature of the invention, the system comprises a cover configured to fit over the support in a form-fitting manner. Advantageously, the cover allows the system to be closed, which reduces any heat exchanges with the outside and promotes temperature maintenance inside the system. Furthermore, the cover allows the plate to be shaped since it is not naturally flat.

According to one feature of the invention, the cover is perforated so that the dispensing head of the automated system can access the wells of the plate.

According to one feature of the invention, the cover comprises at least one notch shaped to engage with a lug of the support. The engagement of the at least one notch with the at least one lug allows the system to be kept closed and also allows the plate to be contained and kept in contact with the cooling module so that the heat exchange is homogeneous and constant in the desired zone. Moreover, the notches extend along the lugs, which allows the cover to be guided when said cover covers the plate.

According to one feature of the invention, the cover comprises at least one protrusion arranged on the face of the cover that is intended to come into abutment on the body of the plate. Advantageously, the at least one protrusion allows the curvature of the plate to be reduced. Advantageously, the at least one protrusion presses on the top of the plate and imposes a flat pressure on the plate by means of the weight of the cover and contact with the cold module.

### BRIEF DESCRIPTION OF THE FIGURES

The invention will be better understood by virtue of the following description, which relates to embodiments according to the present invention, which are provided by way of non-limiting examples and are explained with reference to the appended schematic figures. The appended schematic figures are listed below:

FIG. 1 is a perspective view of the system according to the invention with the plate;

FIG. 2 is another perspective view of the system according to the invention without the cover;

FIG. 3 is a cross-sectional view along a plane P of the system illustrated in FIG. 2;

FIG. 4 is a perspective view of the cooling module according to the invention;

FIG. 5 is a detailed sectional view of the system according to the invention and according to a first embodiment;

FIG. 6 is another detailed sectional view of the system according to the invention and according to a second embodiment;

FIG. 7 is another detailed sectional view of the system according to the invention and according to a third embodiment;

FIG. 8 is another detailed sectional view of the system according to the invention and according to a fourth embodiment;

FIG. 9 is a perspective bottom view of the cover of the system according to the invention;

FIG. 10 is a perspective view of the support of the system according to the invention;

FIG. 11 is a detailed view of the framed ‘A’ portion of FIG. 10.

### DETAILED DESCRIPTION

Irrespective of the embodiment, the system 100 for the automated preparation of a biological sample according to the invention comprises a plate 110, a plate support 120, at least one cooling module 150 configured to at least partially cool each well 111 of the plate 110.

Irrespective of the embodiment, the plate 110 comprises a body 112, from which a plurality of wells 111 extends that are configured to contain a solution that can be a reagent and/or an eluate containing at least nucleic acids. Each well comprises an opening 113 emerging onto the body 112 of the plate 110 and a bottom 114, with each well 111 longitudinally extending in a direction Y-Y′ substantially perpendicular to a direction Z-Z′ along which a surface of the body 112 of the plate 110 extends. As illustrated in FIG. 3, each well 111 comprises a lower portion 111a including the bottom 114 and an upper portion 111b including the opening 113. Irrespective of the embodiment, the cooling module 150 comprises a first part 151 configured to engage with a portion of the plate 110.

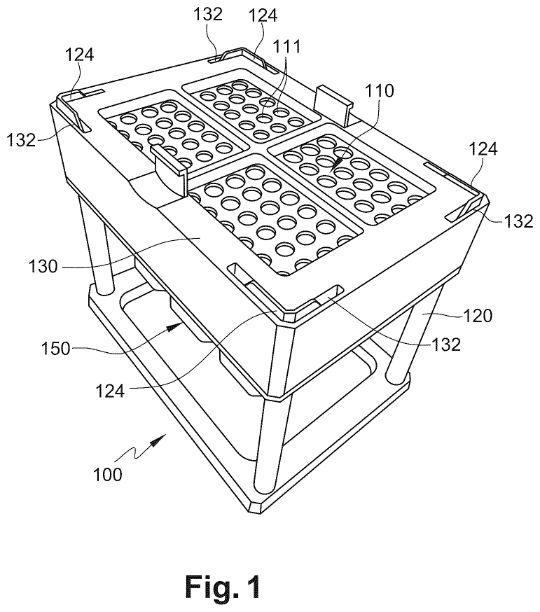

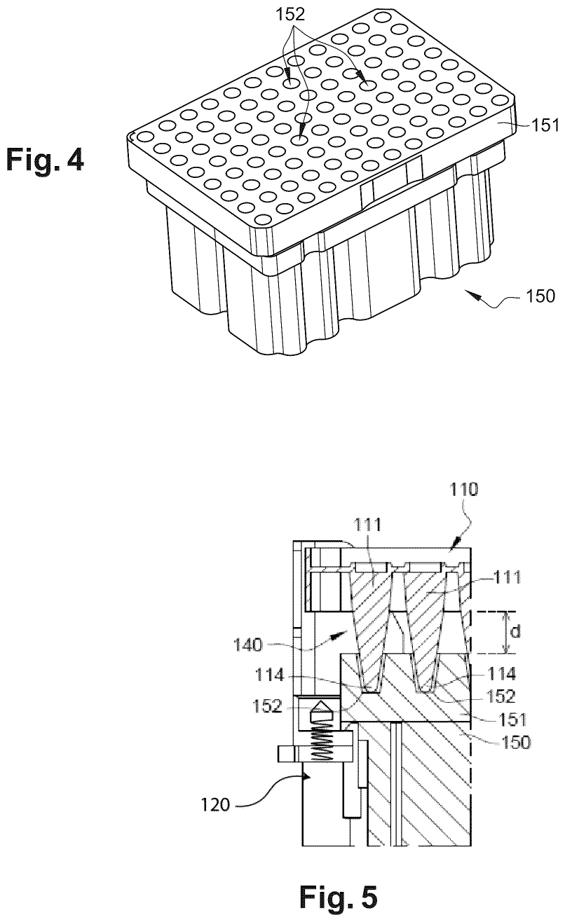

As is notably illustrated in FIGS. 1 and 4, the first part 151 of the cooling module 150 has a plurality of cavities 152 distributed over the surface of the first part 151. Each cavity 152 is shaped in order to partially accommodate a well 111. Indeed, each cavity 152 is shaped in order to accommodate only the lower portion 111a of each well 111, as is notably illustrated in FIG. 3 and in FIGS. 5 to 8. The cavities 152 are evenly distributed over the surface of the first part 151 of the cooling module 150. Preferably, the cooling module 150 comprises as many cavities as the plate 110 has wells 111.

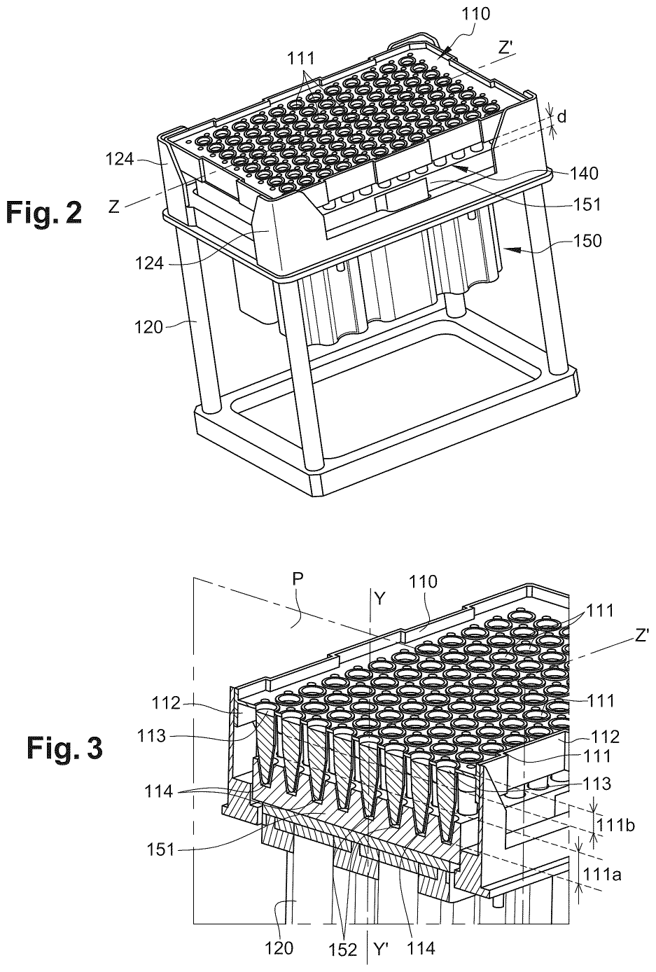

As can be notably seen in FIGS. 2, 3 and 5 to 8, the plate 110 is positioned on the cooling module 150 so that each well 111 of the plate 110 is partially inserted into said cooling module 150.

The body 112 of the plate 110 is at a predetermined distance d from the cooling module 150 so as to delimit a space 140 between said body 112 and the cooling module 150, as can be seen in FIGS. 2, 3 and 5.

Irrespective of the embodiment of the invention, the support 120 comprises a housing 121 configured to receive the plate 110. Advantageously, the housing 121 of the support 120 is configured to at least partially receive the first part 141 of the cooling module 140. As illustrated in FIGS. 3, 10 and 11, the housing 121 comprises a circumferential edge 122, on which the first part 151 of the cooling module 150 rests.

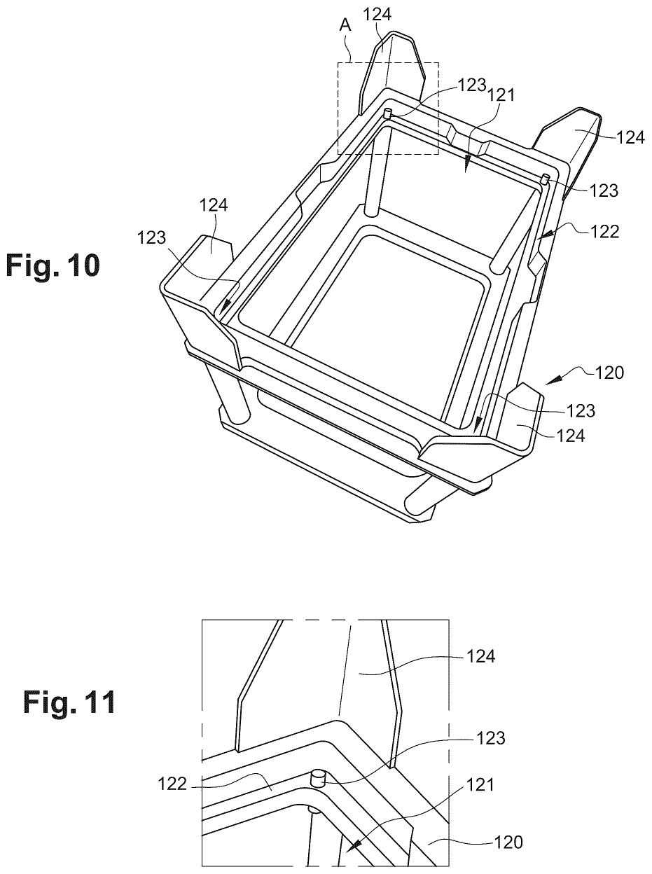

Advantageously, at least one wedging pin 123 is arranged on the circumferential edge 122. As can be seen in FIG. 10, the support 120 comprises four wedging pins 123, thus allowing the cooling module 150 to be evenly adjusted along the Y-Y′ axis.

In the example according to the invention illustrated in the figures, the plate 110 assumes a parallelepiped shape and the housing 121 of the support 120 assumes a complementary shape, so that the circumferential edge 122 of the housing 121 of the support 120 has four corners, with a wedging pin 123 being positioned in each of the corners, as can be seen in detail in FIG. 11.

As is notably illustrated in FIG. 10, the housing 121 of the support 120 further comprises at least one lug 124 in the form of a protuberance extending in a direction perpendicular to the surface of the body 112 of the plate 110. In the illustrated example, the support 120 comprises four lugs 124 each arranged in a corner of the support 120 and being in the form of an extension of a corner of said housing 121 of the support 120. Each lug 124 is configured to engage with the cover 140 of the system 100, as illustrated in FIG. 1.

Irrespective of the embodiment according to the invention, the system 100 comprises a cover 130, as illustrated in FIGS. 1 and 9. As illustrated, the cover 130 assumes a shape complementing that of the support 120 and more specifically assumes a parallelepiped shape. In addition, the cover 130 has a perforated surface for covering the plate 110.

The cover 130 fits onto the support 120. The cover 130 comprises at least one notch 132 configured to engage with a lug 124 of the support 120, as can be seen in FIG. 1. Each lug 124 protrudes relative to the notch 132 that it passes through, as illustrated in FIG. 1. Advantageously, each notch 132 is positioned in a corner of the cover 130, as illustrated in FIG. 1.

Moreover, the cover 130 comprises at least one protrusion 131 arranged on the face of the cover 130 that is intended to come into abutment on the body 112 of the plate 110, as illustrated in FIG. 9. Advantageously, the cover 130 comprises a plurality of protrusions 142 preferably symmetrically distributed along a longitudinal median axis V-V′. Preferably, the cover 130 comprises at least four protrusions 131 distributed over the four corners of the cover 130, even more preferably, five protrusions 131 and even more preferably nine protrusions 131.

FIGS. 5 to 8 will now be described in detail.

According to a first embodiment illustrated in FIG. 5, the space 140 is devoid of any function. According to a variant of the first embodiment, the space 140 contains air or a gas allowing thermal insulation between the body 112 of the plate 110 and the cooling module 150.

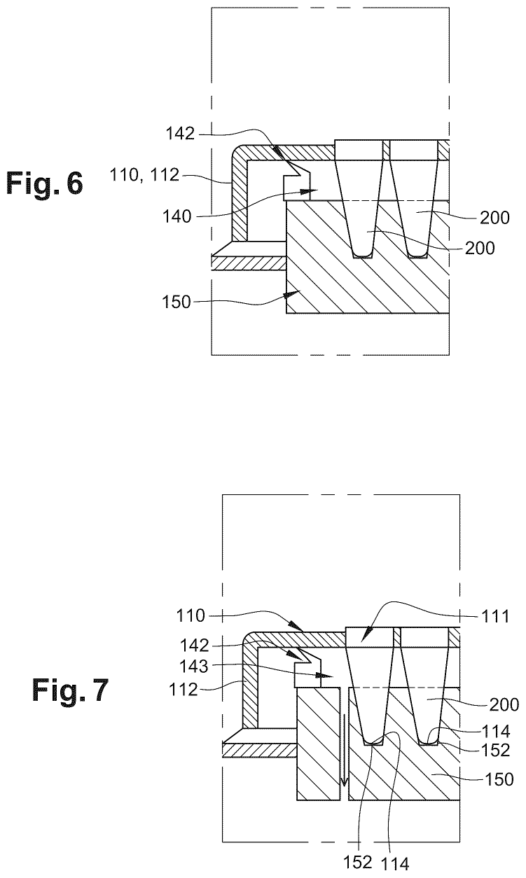

According to a second embodiment illustrated in FIG. 6, the space 140 is provided with a peripheral seal 142 arranged between the cooling module 150 and the body 112 of the plate 110, with the peripheral seal allowing the space 140 to be thermally insulated.

According to a third embodiment illustrated in FIG. 7, the space 140 is provided with a vacuum chamber 143 arranged between the body 112 of the plate 110 and the cooling module 150.

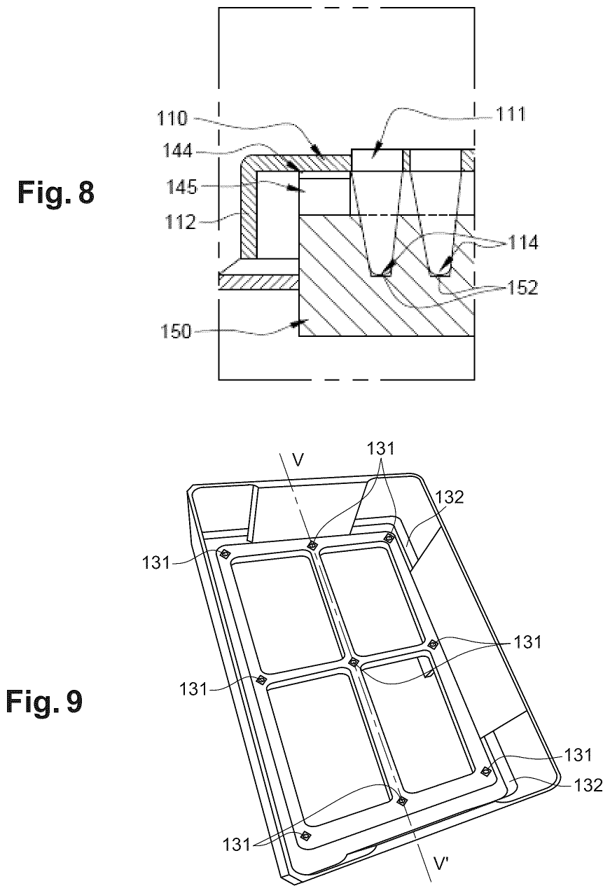

According to a fourth embodiment illustrated in FIG. 8, the space 140 is provided with at least one heating component 144 positioned in the vicinity of the body 112 of the plate 110 and insulated from the cooling module 140 by a thermal insulator 145, which preferably is an insulating foam, for example, made of polymer, for example, of polyethylene, polyurethane, etc., or of elastomer, for example, of nitrile rubber, etc.

As explained, the embodiments differ from one another only in that the space is provided with one or more different elements allowing at least thermal insulation of the body of the plate relative to the lower portion of the wells of the plate.

Of course, the invention is not limited to the embodiments described and shown in the appended figures. Modifications remain possible, notably in terms of the constitution of the various elements or of the substitution of technical equivalents, yet without departing from the scope of protection of the invention.

1. A system for the automated preparation of a biological sample or of a biological mixture comprising at least one reagent and a biological sample or an eluate of the biological sample, comprising at least:
a plate support;
a plate comprising a body, from which a plurality of wells extends that are configured to contain a reagent and/or a biological sample or a biological sample eluate containing at least nucleic acids, each well comprising an opening emerging onto the body of the plate and a bottom, each well longitudinally extending in a direction substantially perpendicular to a direction Z-Z′ along which a surface of the body of the plate extends,
the plate being at least partially housed in the plate support;
at least one cooling module;
wherein the plate is positioned on the cooling module so that each well of the plate is partially inserted into the cooling module, the body of the plate being at a predetermined distance from the cooling module so as to delimit a space between the body of the plate and the cooling module.

2. The system as claimed in claim 1, wherein the space formed between the cooling module and the body of the plate is provided with a thermal insulator.

3. The system as claimed in claim 2, comprising a peripheral seal arranged in the space between the cooling module and the body of the plate.

4. The system as claimed in claim 2, comprising a vacuum chamber arranged between the body of the plate and the cooling module.

5. The system as claimed in claim 2, comprising two distinct zones having a different temperature, the first zone being located between the body of the plate and a thermal insulator, the second zone being located in the vicinity of the cooling module and the temperature in the first zone being higher than the temperature within the second zone.

6. The system as claimed in claim 5, comprising at least one heating component positioned in the vicinity of the body of the plate and insulated from the cooling module.

7. The system as claimed in claim 1, wherein the cooling module comprises a first part configured to engage with a portion of the plate.

8. The system as claimed in claim 7, wherein the first part of the cooling module has a plurality of cavities distributed over the surface of the first part, each cavity being shaped so as to partially accommodate a well of the plate.

9. The system as claimed in claim 1, comprising a cover configured to fit with the support in a form-fitting manner.

10. The system as claimed in claim 9, wherein the cover comprises at least one notch configured to engage with a lug of the support.
